(19) United States
(12) Patent Application Publication
(10) Pub. No.: US 2004/0143294 A1
(43) Pub. Date: Jul. 22, 2004

(54) Septal stabilization device

(75) Inventors: Michael P. Corcoran, Oakdale, MN (US); Joseph A. Marino, Apple Valley, MN (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Cardia, Inc., Burnsville, MN

(21) Appl. No.: 10/349,744

(22) Filed: Jan. 22, 2003

(51) Int. Cl. A61B 17/08

(52) U.S. Cl. 606/213

(57) ABSTRACT

A septal stabilization device for stabilizing a septum. The septal stabilization device comprises right and left fixation devices to which are attached sheets of PVA foam. The fixation devices and sheets limit the motion of the septum and have the added benefit of promoting new tissue growth. The septal stabilization device is sized so that it is large enough to provide stabilization of a septum, yet is not so large that it may cause tissue damage to other portions of the heart.

### CROSS-REFERENCE TO RELATED APPLICATION(S)

This application is related to U.S. patent application entitled Articulated Center Post, Ser. No.______ , U.S. patent application entitled Hoop Design for Occlusion Device, Ser. No.______ , Occlusion Device Having Five or More Arms, Ser. No.______ , and U.S. patent application entitled Laminated Sheets for use in a Fully Retrievable Occlusion Device, Ser. No.______, filed on even date herewith.

### BACKGROUND OF THE INVENTION

The present invention relates to a method and apparatus for stabilizing a septum.

The heart is generally comprised of four chambers, the left and right atrium and the left and right ventricle. Separating the left and right sides of the heart are two walls, or septa. The wall between the two atria is the interatrial septum, and the wall between the two ventricles is the interventricular septum. There are several defects which can affect these septal walls, including patent ductus arteriosus, patent foramen ovale, atrial septal defects (ASDs), and ventricular septal defects (VSDs). A specific type of defect is an ASD or VSD with multiple fenestrations. Multiple fenestrated defects occur when the septum is formed with multiple holes. The multiple holes in the septum lead to residual shunting between the affected chambers of the heart.

In addition, the fossa ovalis region of the atrial septum may experience a localized out pouching. This is termed atrial septal aneurysm (ASA) and causes the septum to “billow” as the heart pumps. An ASA has been identified as a mechanism for neurologic ischemic events, which may lead to transient ischemic attacks (TIAs) or stroke. The exact mechanism of these events is unknown, but a high incidence of ASA has been reported in cryptogenic stroke patients. When other intracardiac or arterial sources of embolism have been ruled out, the detection of an ASA suggests that it may have been the embolic source in patients experiencing stroke or TIA.

Permanently repairing many of these types of septal defects in adults and children used to require open heart surgery, a risky, expensive, and painful procedure. To avoid the risks and discomfort associated with open heart surgery, modern occlusion devices have been developed that are small, illplanltable devices capable of being delivered to the heart through a catheter. There are currently several types of occlusion devices capable of being inserted via a catheter including button devices, collapsible umbrella-like structures, and plug-like devices.

Rather than surgery, a catheter inserted into a major blood vessel allows an occlusion device to be deployed by moving the device through the catheter. This procedure is perforned in a cardiac cathlab and avoids the risks and pain associated with open heart surgery. These modern occlusion devices can be used to treat a wide range of cardiac defects, including patent foramen ovale, patent ductus arteriosus, atrial septal defects, ventricular septal defects. However, the occlusion devices are not suitable for all cardiac problems. For instance, it is difficult to treat ASD or VSD with multiple fenestrations with current occlusion devices.

Furthermore, there are very few suitable methods of treatment for ASAs. Treatment for an ASA may involve placing the patient on anti-coagulants permanently, in an effort to prevent recurrent embolization and subsequent strokes possibly caused by the ASA. Lifetime anticoagulation drug therapies can be expensive, time consuming, difficult to regulate, and may cause other difficulties to the patient. In addition, drug therapies do not stabilize the septum, and have not actually been proven to prevent or reduce the recurrence rate of embolic events or stroke.

Thus, there is a need in the art for minimally invasive treatments for ASAs and other previously hard to treat septal defects.

### BRIEF SUMMARY OF THE INVENTION

The present invention is a method and apparatus for stabilizing a septum. The septal stabilization device comprises a center post, first and second fixation devices, and first and second sheets attached to the first and second fixation devices, respectively. When inserted into the heart, the center post of the septal stabilization device passes through the septum so that the first fixation device and first sheet are on one side of the septum and the second fixation device and second sheet are on the other side of the septum.

The sheets of the device are rigidly held by the center post forcing them into contact with the septal wall. The first and second fixation devices likewise provide structure for holding the sheets against the septal wall. In this manner, the septal stabilization device limits the motion of the septum and has the added benefit of promoting new tissue growth. The septal stabilization device is sized so that it is large enough to provide stabilization of a septum, yet is not so large that it may cause tissue damage to other portions of the heart. Unlike other ASD devices designed to occlude defects, the center post of the septal stabilization device is shortened and need only be long enough to accommodate the septal wall.

The invention further comprises a method of stabilizing an aneurismic or treating an otherwise defective septum. First, a catheter is inserted into the heart and guided to a location of the septum where the septal stabilization device is desired to be deployed. Once in location, the septum is punctured, and the catheter is advanced from a first side of the septum into a second side of the septum. The septal stabilization device is advanced through the catheter so that one side of the device is deployed on the first side of the septum. The catheter is withdrawn through the puncture so that the catheter is located on the second side of the septal puncture. The catheter is then further withdrawn to deploy a second side of the septal stabilization device on the second side of the septum. It is also possible to deploy a septal stabilization device without puncturing the septum by making use of a defect, such as a patent foramen ovale or other ASD.

### BRIEF DESCRIPTION OF THE DRAWINGS

FIG. 1 is a top plan view of the septal stabilization device.

FIG. 2 is a bottom plan view of the septal stabilization device.

FIG. 3 is a side view of the septal stabilization device deployed in a septum.

FIGS. 4A-FIG. 4D illustrates one method of deploying a septal stabilization device.

### DETAILED DESCRIPTION

FIG. 1 is a top perspective view of a septal stabilization device 10. The septal stabilization device 10 comprises a center section 12, a first fixation device 14, and a first sheet 16. The upper fixation device 14 comprises six arms 18 which terminate in atraumatic tips 20. Also shown at the tips 20 are sutures 22. Visible below the first sheet 16 is a second sheet 24.

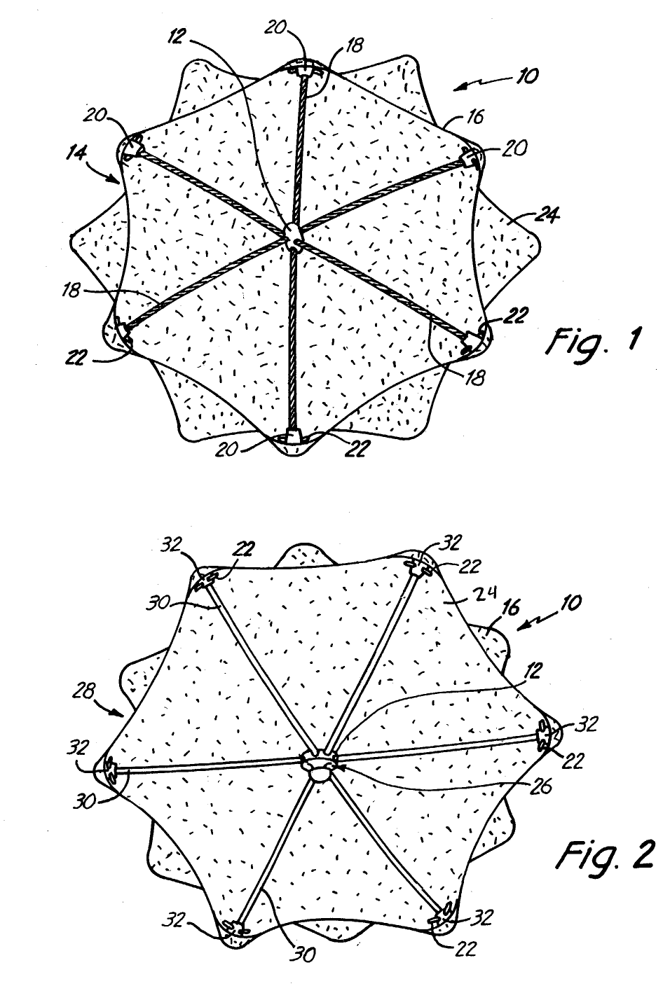

FIG. 2 is a bottom perspective view of the septal stabilization device 10. Visible in FIG. 2 is the center post 12, having at its end a groove 26. Attached to the center post 12 is the second sheet 24 and a second fixation device 28, which is comprised of six arms 30. Similar to the first fixation device 14, the arms 30 of the second fixation device 28 terminate in atraumatic tips 32, and the atraumatic tips 32 further comprise sutures 22.

The first and second fixation devices 14, 28 are connected to the center post 12. One method of connecting the fixation devices 14, 28 to the post 12 is to provide the center post 12 with drill holes through which the fixation devices 14, 28 extend. When connected to the center post 12 using holes drilled through the center post 12, the fixation devices 14, 28 may be formed of three wires. The three wires create the six arms 18, 30 because each wire forms two arms 18, 30 when the wire passes through the center post 12. The atraumatic tips 20, 32 are located at the distal ends of the arms 18, 30 and serve to minimize damage to the surrounding tissue.

The sheets 16, 24 are connected to the septal stabilization device 10 at the center post 12 and at the first and second fixation devices 14, 28. The sheets 16, 24 may be connected to the fixation devices 14, 28 using any suitable method. One method of attaching the sheets 16, 24 to the fixation devices 14, 28 is to suture the sheets 16, 24 to the fixation devices 14, 28 at the atraumatic tips 20, 32. To do so, the atruamatic tips 20, 32 may be provided with drilled holes through which sutures 22 can pass to sew the sheets 16, 24 to the tips 20, 32. Alternatively, the sheets 16, 24 may be sutured to the fixation devices 14, 28 along the length of the arms 18, 30.

The septal stabilization device 10 is configured to be deployed through a catheter, and the groove 26 on the center section 12 is configured to allow the device 10 to be grasped by a forceps as it is guided through the catheter. More specifically, the stabilization device 10 is constructed so that the first and second fixation devices 14, 28 are easily collapsible about the center section 12. Due to this construction, the stabilization device 10 can be folded so that the first fixation device 14 is folded upwards in the axial direction and the second fixation device 28 is folded downwards in the axial direction. The first and second sheets 16, 24 attached to the first and second fixation devices 14, 28 are flexible, and can likewise collapse as the first and second devices 14, 28 are folded.

Once the atrial stabilization device 10 is deployed across a septum, the first and second fixation devices 14, 28 unfold and serve to stabilize the septum and prevent it from billowing. To ensure the atrial septal stabilization device 10 is stiff enough to prevent motion of the septum, the first and second fixation devices 14, 28 are made of a suitable material capable of shape memory, such as nickel-titanium alloy, commonly called Nitinol. Nitinol is preferably used because it is commercially available, very elastic, non-corrosive and has a fatigue life greater than that of stainless steel. To further ensure that the first and second fixation devices 14, 28 do not suffer from fatigue failures, one embodiment of the present invention relies on making the wire fixation devices 14, 28 of stranded wire or cables.

The wire arms 18, 30, are preferably subjected to a precise pre-shaping to give them a “shape memory.” The pre-shaping can be done either by machining, heat treating, or both. The shape memory helps to hold the strands together when the arms 18, 30 are formed of stranded wire or cable, and can be used to add pretension to the arms 18,30 so that they “remember” their shape even after undergoing a strong deformation when the septal stabilization device 10 is passed through a catheter. The atraumatic tips 20, 32 may further serve to prevent potential unraveling of the arms 18, 30 when the arms are formed of stranded wire or cable.

The first and second sheets 16, 24 are preferably formed of a medical grade polymer. One suitable material is DACRON®. Preferably, the sheets 16, 24 are formed of a high density polyvinyl alcohol (PVA) foam, such as that offered under the trade name IVALON®. To minimize the chance of the stabilization device 10 causing a blood clot, the sheets 16, 24 may be treated with a thrombosis-inhibiting material. One such suitable material is heparin. The other parts of the stabilization device 10 are likewise formed of suitable materials. More specifically, the center post 12 may be formed of platinum-iridium, the atraumatic tips 20, 32 may be formed of titanium, and the sutures 22 of polypropylene. However, the invention is not limited to these materials and any suitably biocompatible materials will suffice.

Though not immediately evident in FIGS. 1 and 2, the arms 18, 30 vary slightly in length. This is so that when the device 10 is folded, it fits more easily into a catheter. By making the arms 18, 30 of slightly different lengths, when the device 10 is folded into a catheter, the tips 20, 32 are not all located at the same place. This ensures that when folded, the device 10 is not too bulky to fit into a small diameter catheter.

Though shown with six arms 18, 30, the device 10 is not so limited. Rather, the septal stabilization device 10 may be comprised of four arms, or may be comprised of anywhere from five, six, eight, ten, or even more arms. As the number of arms varies, so too may the shape of the sheets 16, 24. It may be preferable to form the sheets 16, 24 so that the number of sides of the sheets 16, 24 equal the number of arms 18, 30 on the fixation devices. Furthermore, though shown as appearing on the outer sides of the sheets 16, 24, the location of the fixation devices 14, 28 is not so limited. The fixation devices 14, 28 may be located on the outer side of the sheets 16, 24, on the inner side of the sheets 16, 24, or any combination thereof.

As can be seen in FIGS. 1 and 2, the second sheet 24 is offset slightly from the first sheet 16. The degree to which the sheets 16, 24 are offset is variable, and there may be no angle of offset at all. A slight offset between the top sheet 16 and the second sheet 24 is preferred because this means that the fixation devices 14, 28 are likewise offset. Offsetting the fixation devices 14, 28 relative to one another may result in better stabilization of the septum.

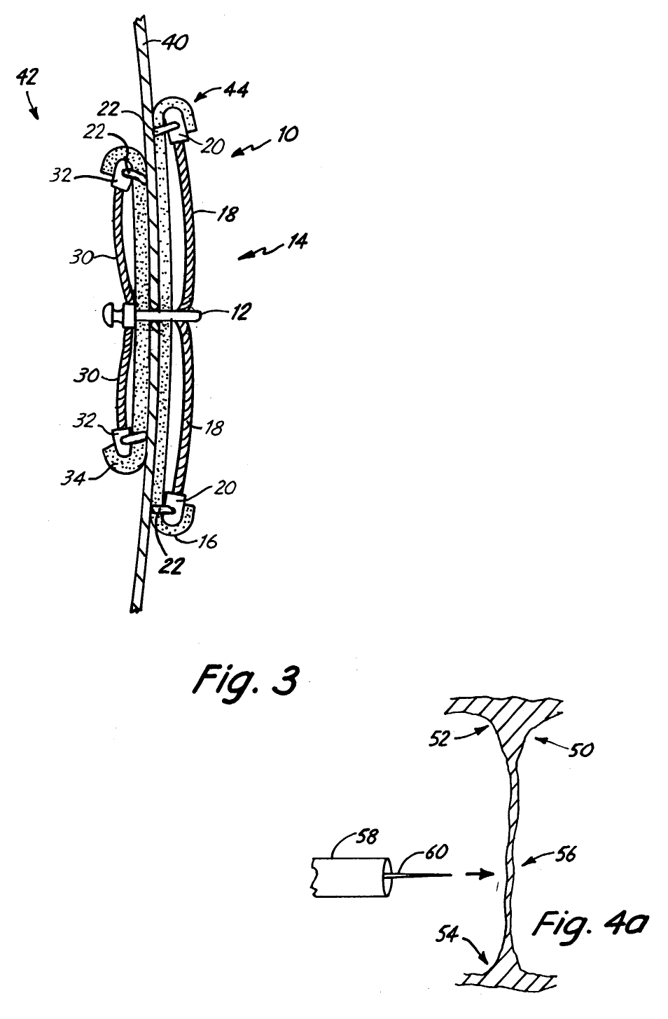

FIG. 3 is a side view of the septal stabilization device deployed across a septum 40. As viewed in FIG. 3, the stabilization device 10 comprises a left side 42 located on a left side of the septum 40, and a right side 44 located on a right side of the septum 40. On the left side 42 is the second sheet 24, and for the sake of clarity, only two arms 30. Also visible on the left side 42 are the tips 32 and the sutures 22 which affix the sheet 24 to the arms 30. On the right side 44 is the first sheet 16, and for the sake of clarity, only two arms 18. Also shown on the right side 44 are the tips 20 and the sutures 22 which affix the sheet 16 to the arms 18. The left side 42 and the right side 44 are connected to the center post 12, which extends through the septum 40.

The shape memory of the arms 18, 30 is more evident in the view allowed in FIG. 3. As can be seen, the arms 18, 30 are shaped such that they urge the sheets 16, 24 toward the septal wall 40. In this manner, the arms 18, 30 extend along the septum 40 to provide a structure which serves to stabilize the septal wall 40, as well as promote tissue growth. The stabilization of the septal wall 40 as well as the additional tissue growth serves to improve the ability of the septal wall to function as normal.

The septal stabilization device 10 does not contain a self-centering mechanism or any mechanical structure in the middle of the device 10. In this way, the septal stabilization device 10 differs greatly from other ASD occluders. For this reason, a septal stabilization device 10 is not suitable for properly deploying in other types of defects. Because the center post 12 does not contain a centering device, the device 10 will typically deploy with the center post 12 biased against an outer margin of the defect. As a result, the septal stabilization device 10 would be located in a significantly offset position in relation to such a defect. In many circumstances, the defect would continue to remain open and the stabilization device 10 would fail to properly occlude the defect. Furthermore, a badly offset position combined with no center structure would make device 10 embolization highly likely if not imminent.

Another difference between the stabilization device 10 and occlusion devices is the reduced length of the center post 12. Generally, other occlusion devices have a center post 12 which may have a length of between about three and about eight millimeters between the sheets 16, 24. The center post 12 of the septal stabilization device 10 is preferably shorter. Preferably, the center post 12 does not contain this length between the sheets 16, 24, but rather places the sheets 16, 24 nearly adjacent each other. This is because there is no need to accommodate the varying topography caused by a septal defect, and the stabilization device 10 need only have a distance between the sheets 16, 24 which is wide enough to accommodate a thin septal wall. However, as discussed below, the center post 12 may require a length when the septal stabilization device is deployed across an existing septal defect, or is deployed at a location on the septum where the setpum is thicker to improve anchoring.

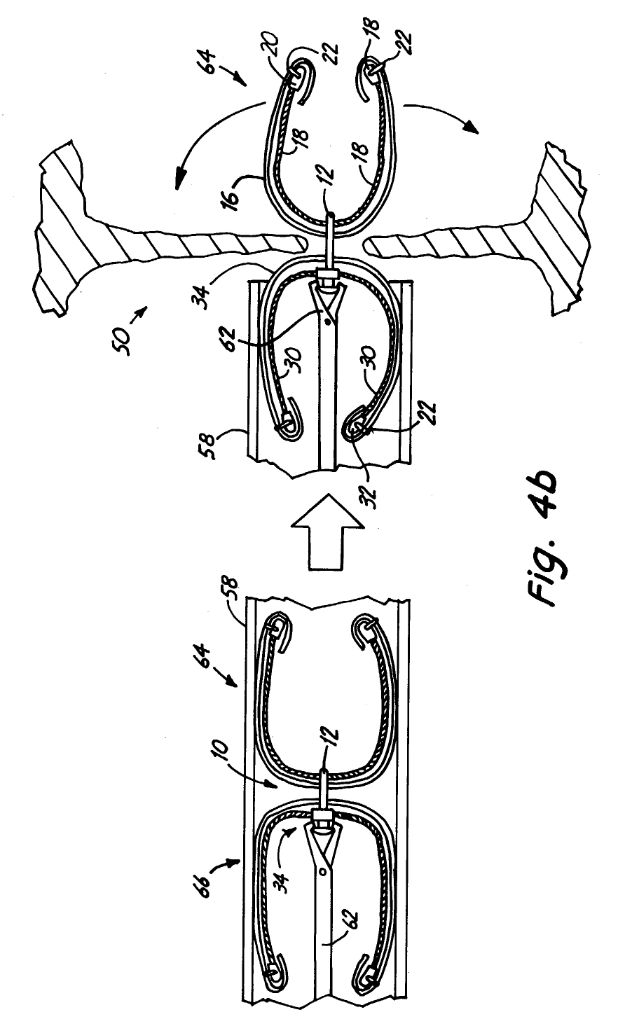

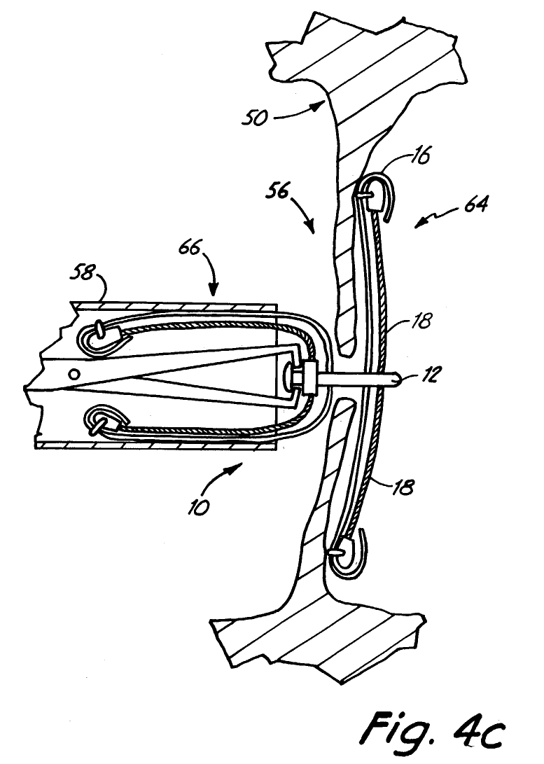

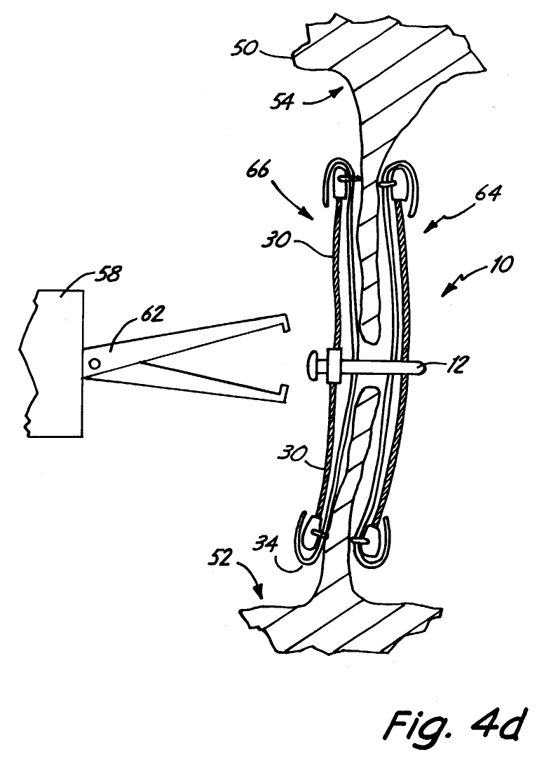

FIGS. 4a-4b illustrate a method of deploying a septal stabilization device across a defective septum. Shown in FIG. 4a is a septum 50 having a thicker top portion 52 and bottom portion 54, but an extremely thin and undeveloped middle portion 56. Also shown in FIG. 4a is a catheter 58 out of which a needle 60 protrudes. As a first step of inserting a septal stabilization device, a needle 60 is used to pierce the septal wall 50 at the desired location. Once the needle 60 has been used to pierce the septum 50, a guide wire may be positioned in the aperture created by the needle 60.

The hole created by the needle 60 must be large enough to expand to the point that the catheter 58 can be passed through it. However, because the tissue of the septum 50 is somewhat elastic, the hole tends to shrink once the catheter 58 is removed. It is preferred that the center post 12 fit fairly snugly in the hole created by the needle 60 after the catheter 58 has been removed.

Care must be taken when puncturing the septum 50 with the needle 60. The puncture must be done carefully to make sure that the needle 60 does not become caught in the septum 50 requiring additional force to either complete the puncture or to remove the needle 60. However, if the needle 60 is driven with too much force, there is a danger that the needle 60 may penetrate surrounding tissue, such as the left atrial wall, causing additional unwanted problems, such as cardiac tamponade. Another danger is dissection of the atrial septum with the needle 60.

Though the needle 60 is shown as piercing the septum 50 at the thin portion 56, it may be more desirable to pierce the septum 50 at the thicker portions 52, 54 to provide a better anchor for the septal stabilization device 10 once it is deployed. Deploying the septal stabilization device 10 where it is more securely anchored, such as in the thicker portions 52, 54, may result in better stabilization of the septum.

FIG. 4b is a side diagrammatic view of a septal stabilization device inserted into the catheter 58. Also shown is a guide forceps 62 which grasps the septal stabilization device 10 at the groove 26 on the center post 12. The guide forceps 62 is used to push the septal stabilization device 10 through the catheter 58 and assist in positioning the septal stabilization device 10 as it is deployed across the defective septum.

As viewed in FIG. 4b, the septal stabilization device 10 comprise a right side 64, a left side 66, and the center post 12. The right side 64 comprises the first sheet 16, arms 18 which terminate in tips 20. At the tips 20 are sutures 22 which attach the sheet 16 to the arms 18. For the sake of clarity, only two arms 18 are shown. Similarly, the left side comprises the second sheet 24, arms 30, tips 32, and sutures 22. For the sake of clarity, only two arms 30 are shown on the left side 66. As described above, the stabilization device 10 is foldable. When inserted into the catheter 58, the right side 64 is folded in a first direction and the left side 66 is folded in a second direction. This allows the arms of the stabilization device 10 to be large enough to stabilize a septum, but also allows the stabilization device 10 to be placed in the small diameter catheter 58.

FIG. 4b illustrates the next step in stabilizing a septum after a hole has been created in the septum 50. The catheter 58 is guided along the guide wire to the location of the needle hole in the septum 50. Once at the hole, the catheter 58 is advanced through the hole in the septum 50 created by the needle 60. After the catheter 58 is positioned in the hole, the guide wire may be removed and the septal stabilization device 10 inserted into the catheter 58. The device 10 is advanced through the catheter 58 using the guide forceps 62. When the device 10 reaches the end of the catheter 58, the guide forceps 62 is used to advance the septal stabilization device 10 out of the catheter 58 so that only the right side 64 is pushed out of the catheter 58.

FIG. 4c shows the next step of placing a stabilization device 10 in a septum 50. After the septal stabilization device 10 is advanced through the catheter 58, the right side 64 is allowed to unfold. In doing so, the arms 18 recover their pre-tensioned shape and hold the sheet 16 against the septum 50. To ensure the stabilization device 10 is properly positioned, the forceps 62 may be used to pull the device 10 snugly against the septal wall 50 as shown in FIG. 4c. Next, the catheter 58 is retracted through the septum 50. Once the catheter 58 is located on the left side of the septum 50, the forceps 62 are used to further advance the device 10 so that the second side 66 of the stabilization device 10 is pushed out of the catheter 58 and unfolds on the other side of the septum 50.

FIG. 4d shows the stabilization device 10 deployed across the septum 50. As shown in FIG. 4d, once the second side 66 is pushed out of the catheter 58, it unfolds and the arms 30 return to their pretensioned shape. The arms 30 hold the sheet 24 against the septum 50. Once the device 10 is positioned as desired, the guide forceps 62 are released from the center post 12 and are withdrawn back through the catheter 58. The catheter 58 is then removed from the body.

In this manner, the septal stabilization device 10 is left in place with the center post 12 extending through the hole created by the needle 60, with one set of fixation devices 64 is on a first side of the septum, and the second set of fixation devices 66 is on the second side of the septum. As described above, the sheets 16, 24 and arms 18, 30 are sized such that once they are deployed on either side of the septum 50, they serve to stabilize the septum and limit its motion. As an added benefit, the sheets 16, 24 and arms 18, 30 promote new tissue growth along the sheets 16, 24 and arms 18, 30 of the devices. This can lead to a further stabilization of the thin portion 56 of the septum 50 because the growth of additional tissue stabilizes the septum.

The placement of the stabilization device 10 is preferably at a location that allows the device 10 to fit snugly against the septum 50 along the thin portion 56 and up to the thicker portions 52, 54. Allowing the arms 18, 30 to contact at least some of the thicker portions 52, 54 of the septum 50 improves the ability of the device 10 to stabilize the septum 50.

This method of stabilizing a septum can be slightly modified in instances where the septum has other types of defects in addition to or in place of an ASA. For instance, if the septum is thin and has a PFO, it may be possible to both close the PFO and stabilize the septum at the same time. In such a case, it may not be necessary to pierce the septum 50 using the needle 60 as illustrated in FIG. 4a. Rather, the device 10 may be deployed across the septal wall 50 through the aperture created by the PFO. Thus, the stabilization device may be deployed across a hole created by the needle, or may be deployed across any other defect which results in connected between chambers separated by the septal wall, such as a hole or flap, which results from an ASD, PFO, or VSD.

In addition, even if there is a PFO or similar septal defect, it may desired to use a needle 60 to pierce the septum 50 regardless of the opportunity to utilize a defect across which to deploy the stabilization device 10. One reason to do so would be to control the location of the septal stabilization device 10. It may be the case that deploying the septal stabilization device 10 at the PFO will not result in the desired amount of stabilization due to the location of the PFO. In such a case, creating the hole through which the stabilization device 10 is deployed may ensure the device is placed at a location of the septum which will provide a better anchor to both close the PFO and stabilization the septum at the same time.

In addition to location, the size of the stabilization device 10 is related to its success at stabilizing the septum 50. Particularly, if a smaller septal stabilization device 10 is deployed at the thin portion of the septum 50, the septum 50 may still “billow” at the thin portion 56 and rather than stabilizing the septum 50, the device 10 may merely billow along with the septum. To prevent this, the septal stabilization device 10 must be sized such that the sheets and upper and lower fixation devices are large enough to provide stabilization of the septum. However, the septal stabilization device 10 must be small enough that it does not interfere with other parts of the heart. If the septal stabilization device is too big, it may start pinching other heart structures or may perforate the tissues of the surrounding structure of the heart. Particularly, when used across the atrial septum, care must be taken to prevent damage to the other heart structures, such as the aorta, that the device may impact.

To accommodate these, it is possible to design the septal stabilization device so that one side of the stabilization device is larger than the other. For instance, one sheet and its associated fixation device can be made smaller than the corresponding sheet and its associated fixation device. This is particularly useful when making one sail smaller allow the stabilization device to be placed at a location in the heart which provides optimal stabilization, without affecting other structures of the heart which may be nearby.

The length of the aneurism and the width of the septum can be used to determine the proper size of the septal stabilization device 10. It is possible to manufacture the septal stabilization device in a plurality of sizes to accommodate various septal lengths and thicknesses. For instance, it is possible to vary the length of the center post 12. Varying the length of the center post 12 refers to varying the distance on the center post 12 between the sheets 16, 24. When deployed at a needle hole created in the septum, the length of the center post 12 is preferably almost zero, and the sheets are placed right next to each other along the center post.

However, if the stabilization device is deployed across an existing defect, or the needle hole is created at a thicker portion of the septum to improve the anchoring of the stabilization device, it is possible to choose a center post length which is greater than zero. In such situations, the physician may choose a length of the center post between about 3 to about 5 millimeters, and in some rare cases up to as high as 8 millimeters.

In addition to choosing the size of the center post, the physician may also choose the size of the sheets 16, 24. The size of the sheets is measured diagonally across the sheet. To ensure the sheets are large enough to provide the desired stabilization, the sheets may be from 18 millimeters to as large as 45 millimeters or more. As described above, it may be desirable to design a stabilization device in which one sheet is of a different size than the other.

Should the stabilization device 10 be placed at a undesired location or experience problems in proper deployment the guide forceps 62 may be used to retrieve the device 10. To retrieve the device 10, the forceps 62 is used to grasp the center post 12 and pull the device 10 back in to the catheter. In doing so, both the right 64 and the left 66 sides of the device will be folded in the same direction inside the catheter 58. To prevent the stabilization device 10 from being too bullky during retrieval due to both sides 64, 66 folding in the same direction, the arms on one side of the device may be made a different length than the arms of the other side. Staggering the length of the arms a small amount ensures that the arms and their atraumatic tips do not become too bulky once the device is retrieved into the catheter.

Though discussed in terms of stabilizing an ASA, the stabilization device 10 is also useful in treating a septum with multiple fenestration. A septum with multiple fenestration occurs when the septum contains a plurality of holes. When using the device to treat a septum with multiple fenestrations, it may be preferable to choose a hole that is somewhat central to the rest of the holes and deploy the septal stabilization device through that centralized hole. As a result of centering the stabilization device in one of the many holes, the arms and sheets are fairly long enough to cover as many of the additional holes as possible, providing for better occlusion of the septum with multiple fenestration. The device also serves to encourage tissue growth, which is further advantageous in treating this type of defect.

In addition, in some septal aneurisms or in some defects with multiple fenestrations, one stabilization device may not suitably occlude all the fenestrations or may not suitably stabilize the septum. The design of the septal stabilization device allows it to be used in connection with another septal stabilization device, or with an occluder. Thus, the septal stabilization device may be deployed to stabilize the septum and occlude some of the multiple fenestrations, while an additional occluder or septal stabilization device is deployed to occlude the remaining fenestrations or provide additional stabilization.

Although the present invention has been described with reference to preferred embodiments, workers skilled in the art will recognize that changes may be made in form and detail without departing from the spirit and scope of the invention. In particular, any of the features disclosed in related applications U.S. patent application entitled Articulated Center Post, Ser. No.______ , U.S. patent application entitled Hoop Design for Occlusion Device, Ser. No.______, Occlusion Device Having Five or More Arms, Ser. No.______, and U.S. patent application entitled Laminated Sheet for use in a Fully Retrievable Occlusion Device, Ser. No.______, filed on even date herewith, may be of use in the present invention. Each of these applications is hereby incorporated by reference.

1. A method of stabilizing a septum, the method comprising:
choosing a location on the septum at which to locate a septal stabilization device;
placing a first part of the septal stabilization device on a first side of the septum at the chosen location; and
placing a second part of the septal stabilization device on a second side of the septum at the chosen location.

2. The method of claim 1 and further comprising:
inserting a catheter into a hole in the septum;
advancing the septal stabilization device through the catheter so that the first part of the septal stabilization device is placed on the first side of the septum;
withdrawing the catheter through hole in the septum; and
advancing the septal stabilization device through the catheter so that the second part of the septal stabilization device is placed on the second side of the septum.

3. The method of claim 2 and further comprising creating the hole in the septal wall at the chosen location.

4. The method of claim 2 and further comprising choosing a size of the septal stabilization device that ensures the septal stabilization device will prevent atrial septal aneurism.

5. The method of claim 1 and further comprising occluding a septal defect.

6. The method of claim 1 and further comprising choosing a size of the septal stabilization device.

7. The method of claim 6 wherein choosing a size of the septal stabilization device comprises choosing a size of a center post of the stabilization device.

8. The method of claim 6 wherein choosing a size of the septal stabilization device comprises choosing a size of the first and second parts.

9. A method of stabilizing a septum, the method comprising:
inserting a catheter into the heart;
positioning the catheter through a hole in a septal wall;
deploying a first side of a septal stabilization device on a first side of the septal wall;
withdrawing the catheter through the hole in the septal wall; and
deploying a second side of the septal stabilization device on a second side of the septal wall; wherein the first and second sides of the septal stabilization device are sized to allow them to stabilize the septum.

10. The method of claim 9 and further comprising:
creating the hole in the septal wall through which the catheter is positioned.

11. The method of claim 10 and further comprising:
placing a guide wire at the hole;
advancing the catheter along the guide wire until the catheter is positioned in the hole; and
removing the guide wire.

12. The method of claim 9 wherein positioning the catheter through a hole in the septal wall comprises positioning the catheter in a pre-existing septal defect.

13. The method of claim 9 wherein the pre-existing septal defect comprises a patent foramen ovale.

14. The method of claim 9 wherein the pre-existing septal defect comprises a septum with multiple fenestration.

15. The method of claim 9 wherein deploying the septal stabilization device on the first side of the septal wall and on the second side of the septal wall further serves to occlude a septal defect.

16. The method of claim 9 and further comprising deploying a second stabilization device.

17. The method of claim 9 and further comprising deploying an occlusion device.

18. A septal stabilization device, the septal stabilization device comprising:
a center post;
a first fixation device connected to the center post;
a second fixation device connected to the center post; and
first and second sheets attached to the first and second fixation devices, wherein the first and second sheets are held along the septum by the first and second fixation devices and serve to stabilize the septum.

19. The septal stabilization device of claim 18 wherein the first and second sheets comprise polyvinyl alcohol foam.

20. The septal stabilization device of claim 18 wherein the first and second fixation devices comprises stranded wire support arms.

21. The septal stabilization device of claim 18 wherein the first and second fixation devices comprise six arms.

22. The septal stabilization device of claim 18 wherein the first and second fixation devices are attached to the center post directly adjacent one another so that there is no distance on the center post between the first and second fixation devices.

23. The septal stabilization device of claim 18 wherein the distance on the center post between the first and second fixation devices is less than about 3 millimeters.

24. The septal stabilization device of claim 18 wherein the center post comprises a groove configured to be grasped by a guide forceps.

25. The septal stabilization device of claim 18 wherein the first and second fixation devices and the first and second sheets are configured to allow the device to be folded and inserted into a catheter.

26. The septal stabilization device of claim 18 wherein the first and second sheets are connected to the first and second fixation devices with sutures.

27. The septal stabilization device of claim 26 wherein the first and second fixation devices further comprise atraumatic tips and the first and second sheets are connected to the first and second fixation devices using sutures at the tips.

28. The septal stabilization device of claim 26 wherein the size of the first and second sheets is between about 18 and about 45 millimeters.

29. The septal stabilization device of claim 28 wherein a size of the first sheet is not equal to a size of a second sheet.
